(19) United States
(12) Patent Application Publication
(10) Pub. No.: US 2016/0106412 A1
(43) Pub. Date: Apr. 21, 2016

(54) Method of Performing a Mastopexy Procedure

(71) Applicants: Peter Petros, Elizabeth Bay (AU); Paul Zadow, Allenby Gardens (AU)

(72) Inventors: Peter Petros, Elizabeth Bay (AU); Paul Zadow, Allenby Gardens (AU)

(21) Appl. No.: 14/868,986

(22) Filed: Sep. 29, 2015

(30) Foreign Application Priority Data
Oct. 17, 2014 (AU) 2014904161

(51) Int. Cl. A61B 17/04

(52) U.S. Cl. CPC A61B 17/0401; A61B 2017/00792

(57) ABSTRACT

The present invention relates to a method of performing a mastopexy procedure. The method includes the steps of:
a) inserting a filament into breast tissue of a patient's breast;
b) anchoring said filament at a first location and a second location so that the filament extends between the first and second locations; and
c) positioning said filament within the breast tissue so that the filament between the first and second locations acts as a stay supporting at least a part of the weight of the breast.

### TECHNICAL FIELD

The present invention relates to a method of performing a mastopexy procedure. Mastopexy is a surgical procedure performed to correct ptosis or sagging of the breasts. The procedure may have application in both male and female patients.

### BACKGROUND OF INVENTION

Mastopexy, sometimes more commonly referred to as breast lift, is a surgical procedure performed to raise and reshape sagging breasts. It is a procedure commonly conducted on woman whose breasts lose their shape and firmness due to the impact of gravity, aging, pregnancy, breast feeding, or weight changes.

Mastopexy may be conducted in conjunction with a breast augmentation procedure. Such procedures may involve the introduction of a breast implant to increase breast size.

Mastopexy is not a permanent solution to sagging breasts. Over time the breasts will continue to sag as a result of aging, hormonal changes and the impact of gravity. Accordingly, mastopexy patients need to understand that the procedure will only raise and reshape their breasts for some period of time. The extent of that time period will depend on many factors that will vary from patient to patient. Thus, a patient may need to have the procedure repeated to maintain the youthful breast appearance they are seeking.

The present invention seeks to provide a method of performing a mastopexy procedure that prevents or at least delays further sagging of the breasts.

The discussion of the background to the invention herein is included to explain the context of the invention. This is not to be taken as an admission that any of the material referred to was published, known or part of the common general knowledge as at the priority date of this application.

### SUMMARY OF INVENTION

According to a first aspect of the present invention there is provided a method of performing a mastopexy procedure including the steps of:
a) inserting a filament into breast tissue of a patient's breast;
b) anchoring said filament at a first location and a second location so that the filament extends between the first and second locations; and
c) positioning said filament within the breast tissue so that the filament between the first and second locations acts as a stay supporting at least a part of the weight of the breast.

Preferably, a part of the filament extends through the breast tissue below the tissue of the areola and substantially in line with the patient's nipple, or below the patient's nipple, or above the patient's nipple.

In accordance with an embodiment of the invention, the first location is in the pectoralis major and the second location is in the pectoralis major. The second location is between about 4-10 cm horizontally from the first location.

Once the filament has been positioned within the breast tissue and it is determined that the filament between the first and second locations is tensioned to support the breast as required by the surgeon, the filament is preferably secured to prevent any inadvertent increase in the length of filament between the first and second locations.

It is envisaged that different forms of filament may be used in a method in accordance with an embodiment of the invention. However, the filament is preferably a tape which is formed of a knitted or woven material. The material is preferably selected so that once the material is located within the breast tissue it will facilitate the growth of scar-like tissue into the filament.

The filament is preferably anchored at each of the first and second locations using respective anchors. The anchors are preferably as described in international patent application PCT/AU2003/001036 (published as WO 2004/017845).

Embodiments of the first aspect of the invention are advantageous because the filament works like a conventional brassiere to support at least a part of the weight of the breast tissue, preventing or reducing further elongation of the breast due to gravity.

According to a second aspect of the invention there is provided a method of performing a mastopexy procedure including the steps of:
a) making a first incision along a first incision line at or adjacent to the perimeter of the patient's areola;
b) making a second incision along a second incision line spaced from the first incision so that an area of skin is defined between the first and second incisions;
c) removing the epithelial layer from a donut shaped part of the area of skin; and
d) suturing the second incision line to the first incision line.

The area of skin defined between the first and second incisions is preferably donut shaped. However it is envisaged that other shapes of skin between the first incisions and second incisions are possible. Where the area of skin between the first and second incisions adopts a shape other than donut shaped, it is envisaged that the epithelial layer would only be removed from a donut shaped part of that area of skin.

Preferably, the donut shape area of skin is folded over on itself, at least in part, when the second incision line is sutured to the first incision line. This effectively increases the volume of tissue around the nipple, restoring the shape with a very natural appearance. Importantly, the breast remains soft because an implant is not required to achieve enhancement.

In accordance with a preferred embodiment of the invention, the epithelial layer from the donut shaped area of skin is removed with a dermatome. The dermal layer is preferably not removed from the donut shaped area of skin.

The first and second incisions are preferably made to a depth sufficient to penetrate the Cooper's ligaments. Typically, this requires an incision depth of about 1.5 cm.

According to a third aspect of the present invention there is provided a method of performing a mastopexy procedure that combines the steps of the first and second aspects of the invention. Such a method is particularly advantageous because it combines the benefits of both the first and second aspects of the invention.

In accordance with an embodiment of the third aspect of the invention, the filament is preferably inserted into the breast tissue via a location within the donut shaped part of the area of skin. The length of filament between the first and second locations can preferably be reduced during the procedure to provide a required degree of tension in the filament. Preferably this is achieved by pulling one end of the filament through one of the first or second anchors.

### BRIEF DESCRIPTION OF DRAWINGS

Embodiments of the invention will now be described, by way of example only, with reference to the accompanying drawings in which:

FIG. 1 is an isometric view of an anchor and filament suitable for use in a mastopexy procedure in accordance with a method of an embodiment of the invention;

FIG. 2 is an isometric view of a length of filament and two anchors. The anchors are each mounted in a holder;

FIG. 3 is a view similar to FIG. 2, but showing a first anchor applicator connected to the first holder;

FIGS. 4a and 4b illustrates respective front and cross-sectional views of a patient showing the location of the anchors and filament in accordance with a mastopexy method according to an embodiment of the invention;

FIG. 4c is a front view of a patient with incisions marked and the position of the anchors and untightened filament shown schematically;

FIG. 5 schematically illustrates the incisions made during a mastopexy method according to an embodiment of the invention; and

FIG. 6 schematically illustrates a cross-section of the breast following a mastopexy procedure using the method of an embodiment of the invention. One anchor and a part of the filament are depicted.

### DETAILED DESCRIPTION

The actual incisions performed by a surgeon during a conventional mastopexy procedure will depend on factors such as the size of the breast and the amount of lift that is needed. The configuration of the different incisions provide the names for the most common conventional breast lifts and these include the “anchor” lift, the “lollipop lift”, the “crescent” lift and the “donut” lift. It is envisaged that the present invention may have application with any of these different breast lifts or to any variations thereof. However, for the purpose of the description of an embodiment of the invention, a breast lift conducted with a “donut” incision, sometimes referred to as a Benelli lift, will be described.

Prior to surgery, the patient will typically undergo an examination and the surgeon will determine and mark on the patient's breasts the proposed incision lines to achieve the required lift. As explained below, these proposed incision lines may be adjusted during the procedure.

The “donut” lift requires the surgeon to make a first incision around the perimeter of the areola. The first incision may be made inwardly of the external perimeter of the areola where a reduction of areola size is also desired. A second circular incision is then made spaced from the first incision so that the two incisions define a “donut” shaped area of skin. The incisions are made full thickness so as to penetrate the Cooper's ligaments of the breast (e.g. about 1.5 cm deep). Once the incisions have been made, the epithelial layer of the donut shaped area of skin between the two incisions is removed. Different skin removal techniques are envisaged, for example, the use of a dermatome to remove only a superficial layer of skin (i.e. the epithelial layer) from the donut shaped area. Whatever method used, the surgeon should ensure that only the epithelial layer is removed, leaving the dermis intact.

Once the epithelial layer from the donut shaped area has been removed, a filament is inserted into the breast tissue and supported from the pectoralis major fascia at two points to establish a U-shaped filament configuration. As will be explained in more detail below, once the surgical procedure is completed, the filament acts as an internal support or stay within the breast to prevent further descent or sagging of the lifted breast. The second incision line is then sutured to the first incision line causing the donut shaped area (with epithelial layer removed) to be folded to form what is described as a ‘double layer’ of tissue around the areola. This ‘double layer’ bulks up the area surrounding the areola.

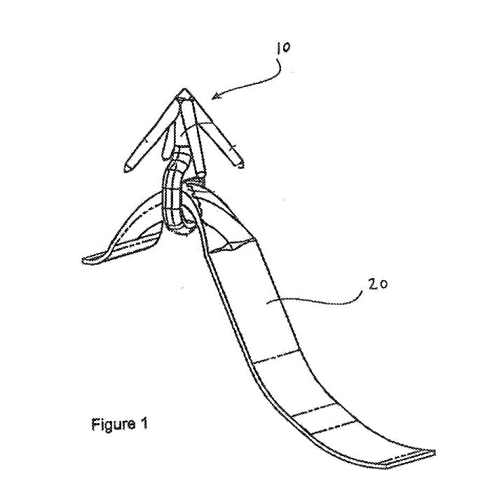

It is envisaged that the filament would be anchored to the pectoralis major fascia using two anchoring devices and a filament of the type as generally described in international patent application PCT/AU2003/001036 (published as WO 2004/017845), the contents of which are incorporated herein by reference. FIG. 1 illustrates such an anchor 10 with a length of filament 20 passing through the anchor 10.

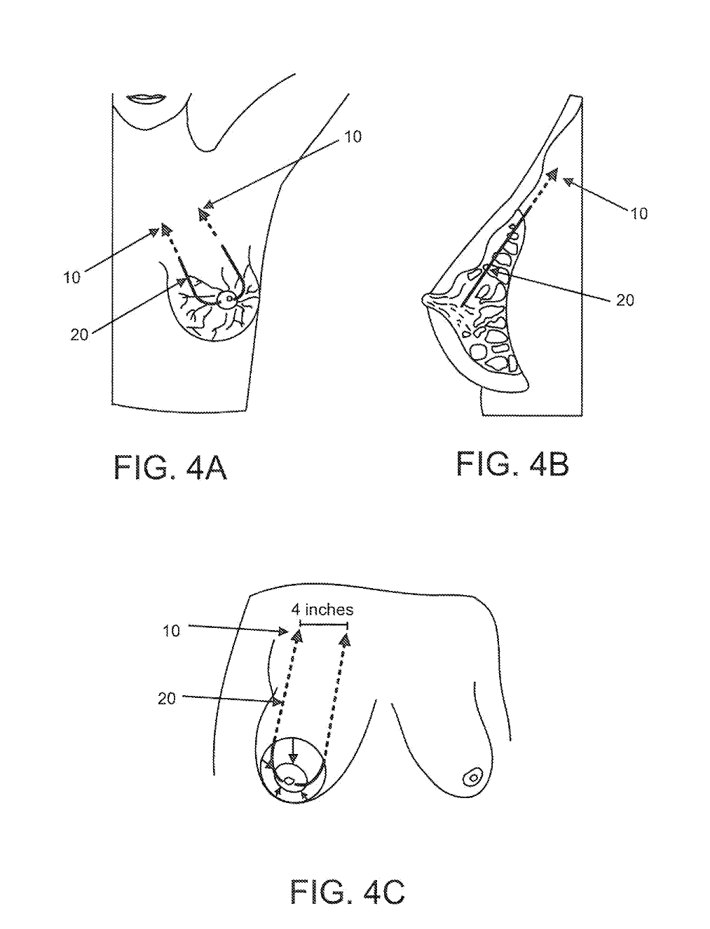

FIGS. 4a and 4b schematically illustrates two anchors 10 and a filament 20 connected therebetween inserted within a patient who has undergone a mastopexy procedure according to an embodiment of the invention. Although exact patient positioning may vary, it is envisaged that the anchors 10 would enter the pectoralis major fascia at about 4 cm below the patient's clavicle. As the breasts sag laterally, the filament 20 is arranged to retract the breast towards the medial part of the patient's chest. The horizontal spacing between the anchors 10 would again vary from patient to patient but it is envisaged that a minimum appropriate spacing would be about 4 cm. A maximum appropriate spacing would be about 10 cm, with the median distance varying between about 6 cm and about 10 cm.

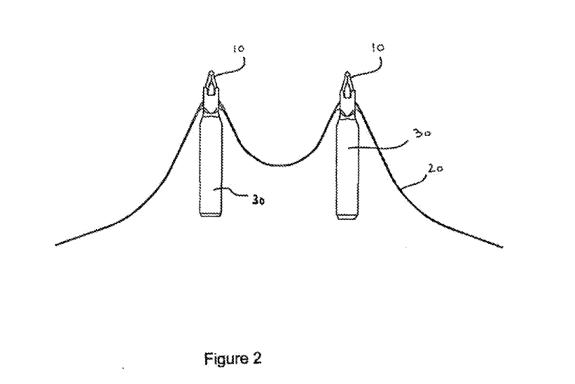

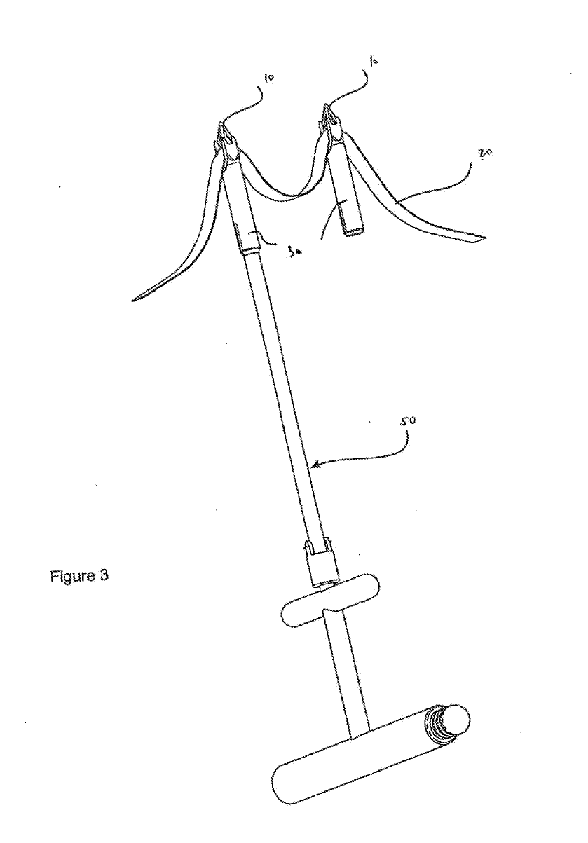

The steps of a method according to an embodiment of the invention are preferably as follows:
1. The breast is marked with a first incision line around the areola or inwardly of the circumference of the areola if reduction in areola size is required. A second incision line is then marked spaced from the first incision line defining a donut shaped area of skin between the first and second marked incision lines.
2. The second marked incision line is then sutured to the first marked incision line at four points around the circular marked incision line so that the surgeon can assess likely appearance of the breast following completion of the procedure. If the surgeon is not satisfied with the appearance, the position of the marked second incision line can be varied so as to increase or decrease the ‘double layer’ of tissue that will later be established around the areola. The surgeon repeats this step until satisfied that the breast following the procedure will have an acceptable appearance.
3. Incisions are then made along the first and second incision lines. The incisions are made full thickness to penetrate the Cooper's ligaments, preferably to a depth of about 1.5 cm.
4. The epithelial layer of skin between the first and second incisions is then removed.
5. Metzenbaum scissors are inserted laterally about 1.5 cm below the skin through the area where the epithelial layer of skin has been removed. The scissors are directed towards the second rib to penetrate the collagenous fascial covering of the pectoralis major at about the level of the second rib. The fascia is penetrated and the scissors proceed towards the clavicle for about 4 cm.
6. The surgeon selects a length of filament 20 on which is located first and second anchors 10. As shown in FIG. 2, each anchor 10 is mounted within a holder 30. In accordance with this embodiment, the holder 30 is configured to hold the prongs inwardly of the anchor 10 so that the prongs cannot engage with the patient's tissue. It is of course envisaged that the prongs may be held, located or otherwise configured to ensure that the prongs do not engage during this step with the patient's tissue.
7. An anchor applicator 50 is loaded with the holder 30 containing the first anchor 10. The anchor applicator 50 is used to locate the exposed tip of the first anchor 10 into the site previously penetrated by the scissors. The anchor applicator 50 is held in place at the first site.
8. The part of the filament 20 with the second anchor 10 (housed within the second holder 30) is brought downwardly to touch the surface of the donut shaped tissue denuded of the epithelial layer. Scissors are then used to form a hole below the tissue of the areola and generally in line with the nipple so that the second anchor 10 and filament 20 can be passed through that hole to exit the distal side of the areola (see FIG. 4b). In other embodiments, the hole may be located below the nipple or above the nipple.
9. Metzenbaum scissors are then inserted into the fascia covering the pectoral is major proceeding for about 4 cm at a distance of about 8-10 cm from the first anchor 10 to expose a second site. An anchor applicator 50 is loaded with the second holder 30 containing the second anchor 10. The second anchor applicator 50 is used to locate the exposed tip of the second anchor 10 into the second site. The second anchor applicator 50 is held in place at the second site.
10. The first and second incision lines are then approximated with Allis forceps to re-check the sufficiency of the donut shaped area of skin. Interrupted sutures are then placed at 12, 6, 3 and 9 o'clock positions to temporarily hold the breast in the lifted position.
11. The length of filament 20 between the first and second anchors 10 is then tightened just enough so that the filament 20 supports the lifted breast without creating a ridge in the skin. The filament 20 should be tightened just sufficiently to hold the lifted breast in place, but not to actually elevate it any further. The filament 20 is intended to act much like the straps of a brassiere by providing support to the breast tissue.
12. If the surgeon is not satisfied with the resultant appearance, the length of filament 20 can be loosened between the first and second anchors 10 and then the sutures and forceps removed. The positioning of one or both of the anchors 10 can then be adjusted by adjusting the positioning of the holders 30. Steps 10 and 11 can then be repeated.
13. When the surgeon is satisfied with the appearance of the lifted breast, each anchor applicator 50 is used to firmly secure the respective anchor 10 into the required site. Each anchor applicator 50 with holder 30 is then removed from the patient.
14. The lateral ends of the filament 20 are sutured to the medial part of the filament 20 to prevent later slippage of the filament 20 through the anchors 10. The excess lateral ends of the filament are trimmed.
15. The second skin incision is then properly sutured to the first skin incision.
16. Steps 1-15 are then preferably repeated on the second breast.

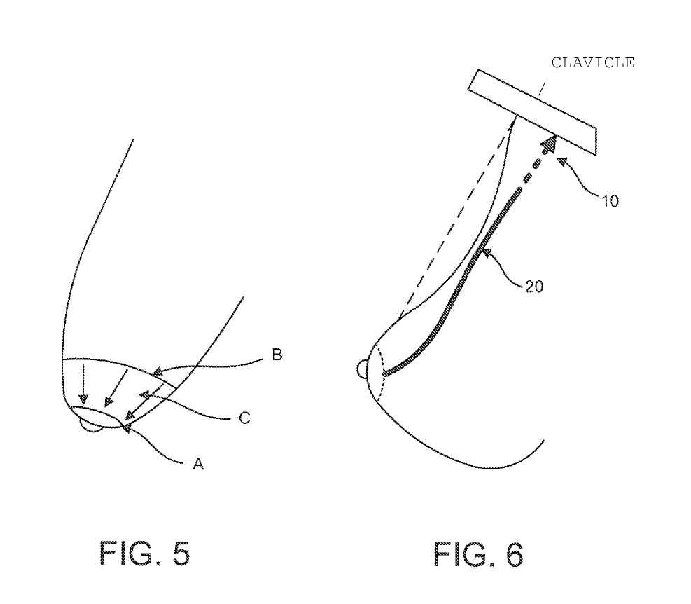

FIG. 5 schematically illustrates a breast with a first incision line A around the perimeter of the areola and a second incision line B spaced from the first incision line A. The donut shaped area C bordered by the first and second incision lines A, B is marked with arrows. The arrows signify how the second incision line B is brought towards the first incision line A to achieve breast lift, folding over the intervening tissue. As explained above, sutures are then applied to join the first and second circular incision lines A, B together. In doing this, the remaining tissue of the donut shaped area C (which includes remnants of Cooper's ligaments) forms the ‘double layer’ to bulk up the atrophied glandular layer around the nipple. That tissue is buried below the skin augmenting the breast volume and providing a rounder and more youthful breast shape. The patient's nipple is lifted upwardly above the level of the inframammary fold, improving the slope of the breast (see the dashed line in FIG. 6). It is noted that only the epithelial layer has been removed from the donut shaped area C between the first and second incision lines A, B.

As schematically shown in FIGS. 4 and 6, once the mastopexy procedure is complete, the filament 20 is located underneath the mid-part of the patient's nipple and acts to prevent further sagging of the breast. It can be seen by comparing FIGS. 5 and 6 that the slope of the breast (indicated by the dashed lines) has been improved by the procedure.

Over time, fibrosis of the filament 20 will occur so that the filament acts as a large Cooper's suspensory ligament to prevent further ptosis of the breast.

Following the procedure, if the slope of the breast requires attention, the skin below the patient's clavicle can be incised or excised and re-attached to the fascia below the clavicle line. A crescent shaped incision or excision would be made to shorten the skin and to re-attach it to the clavicular fascia.

Embodiments of the invention according to the first and third aspects of the invention are advantageous because the insertion of the filament within the lifted breast acts as an internal brassiere supporting the weight of the breast tissue against the impact of gravity. The internal stay or brassiere established by the filament helps to prevent or at least delays further breast ptosis.

It will be appreciated by a person skilled in the art that the above procedure is advantageous because the filament works like a brassiere to support the weight of the breast tissue, preventing further elongation due to gravity.

It will further be appreciated that embodiments according to the second and third aspect of the invention increase the volume of tissue around the nipple, restoring the shape with a very natural appearance. Importantly, the breast remains soft whilst still providing increase volume of tissue around the nipple. In contrast, prior art procedures excise the tissue in this area full thickness (i.e. removal of both the epithelial and dermal layers) and then use an implant to establish increased volume (i.e. increased breast size). This is a more invasive procedure as it typically requires cutting and dislocation of the pectoralis major fascia to locate the implant. Although the implant provides the necessary enhancement, the appearance is not natural and the implant soon fibroses, causing the tissue to become hard and unnatural in feel. Many patients are keen to avoid the use of implants when and if possible.

Embodiments of the present invention are further advantageous as they result in the patient's nipple, which was previously facing vertically downwards, being rotated and elevated so at to extend generally horizontally outwardly.

The embodiments have been described by way of example only and modifications within the spirit and scope of the invention are envisaged.

1. A method of performing a mastopexy procedure including the step of:
a) inserting a filament into breast tissue of a patient's breast;
b) anchoring said filament at a first location and a second location so that the filament extends between the first and second locations; and
c) positioning said filament within the breast tissue so that the filament between the first and second locations acts as a stay supporting at least a part of the weight of the breast.

2. A method according to claim 1 wherein a part of the filament extends through the breast tissue below the tissue of the areola and substantially in line with the patient's nipple, or below the patient's nipple, or above the patient's nipple.

3. A method according to claim 1 or claim 2 wherein the first location is in the pectoralis major muscle and the second location is in the pectoralis major muscle.

4. A method according to claim 3 wherein the second location is between about 4-10 cm horizontally from the first location.

5. A method according to claim 1 further including:
a) making a first incision along a first incision line at or adjacent to the perimeter of the patient's areola;
b) making a second incision along a second incision line spaced from the first incision so that an area of skin is defined between the first and second incisions;
c) removing the epithelial layer from a donut shaped part of the area of skin; and
d) suturing the second incision line to the first incision line.

6. A method according to claim 5 wherein the donut shaped part of the area of skin is folded over on itself at least in part when the second incision line is sutured to the first incision line.

7. A method according to claim 5 wherein the epithelial layer from the donut shaped area of skin is removed with a scalpel and/or with a dermatome.

8. A method according to claim 5 wherein the filament is inserted into the breast tissue at a location within the donut shaped area of skin.

9. A method according to claim 5 wherein the first and second incisions are made to penetrate the Cooper's ligaments.

10. A method according to claim 5 wherein the first and second incisions are made to a depth of about 1.5 cm.

11. A method according to claim 5 further including the steps of:
a) inserting a filament into breast tissue of a patient's breast;
b) anchoring said filament at a first location and a second location so that the filament extends between the first and second locations;
c) positioning said filament within the breast tissue so that the filament between the first and second locations acts as a stay supporting at least a part of the weight of the breast.

12. A method according to claim 11 wherein the filament is inserted into the breast tissue via a location within the area of skin.

13. A method according to claim 11 further including the step of securing the filament so that the length of filament between the first and second locations is fixed to maintain a required degree of tension in the filament.

14. A method according to claim 1 or claim 11 wherein the length of filament between the first and second locations can be reduced during the procedure to provide a required degree of tension in the filament.

15. A method according to claim 5 wherein the area of skin is donut shaped.

16. A method of performing a mastopexy procedure including the steps of:
a) marking the breast with a first incision line around the areola or inwardly of the circumference of the areola;
b) marking the breast with a second incision line spaced from the first incision line defining a donut shaped area of skin between the first and second marked incision lines;
c) suturing the second marked incision line to the first marked incision line so that the surgeon can assess likely appearance of the breast following completion of the procedure;
d) incising along the first and second incision lines;
e) removing the epithelial layer of skin between the first and second incisions;
f) exposing a first location and locating a tip of an anchor at the first location, the anchor connected to a filament;
g) forming a hole below the tissue of the areola so that a second anchor and a part of the filament can be passed through that hole to exit the distal side of the areola;
h) exposing a second location and locating a tip of the second anchor at the second location;
i) approximating the first and second incision lines to check the sufficiency of the donut shaped area of skin;
j) placing sutures to the first and second incision lines to temporarily hold the breast in the lifted position;
k) adjusting the length of filament between the first and second anchors so that the filament supports the lifted breast without creating a ridge in the skin;
l) securing the first and second anchors into the first and second locations;
m) securing the filament to prevent later slippage of the filament that would vary the length of filament between the first and second anchors;
n) suturing the second incision to the first incision.

17. A method according to claim 16 further including repeating the method on the patient's second breast.

18. A method according to claim 16 or claim 17 wherein if the surgeon is not satisfied with the appearance of the breast on completion of step i), the position of the marked second incision line is varied.

19. A method according to claim 16 wherein the incisions are made full thickness to penetrate the Cooper's ligaments.

20. A method according to claim 16 wherein the epithelial layer is removed using a dermatome.

21. A method according to claim 16 wherein if the surgeon is not satisfied with the appearance of the lifted breast on completion of step k), the length of filament can be loosened between the first and second anchors, the temporary sutures removed and the positioning of one or both of the anchors adjusted.

22. A method according to claim 16 wherein the hole below the tissue of the areola is formed generally in line with the patient's nipple, or above the patient's nipple or below the patient's nipple.

23. A method of performing a mastopexy procedure including the steps of:
a) making a first incision along a first incision line at or adjacent to the perimeter of the patient's areola;
b) making a second incision along a second incision line spaced from the first incision so that an area of skin is defined between the first and second incisions;
c) removing the epithelial layer from a donut shaped part of the area of skin; and
d) suturing the second incision line to the first incision line.

24. A method according to claim 23 wherein the area of skin is donut shaped.
